(19) United States
(12) Patent Application Publication
(10) Pub. No.: US 2023/0081502 A1
(43) Pub. Date: Mar. 16, 2023

(54) SPECTRAL SATURATION IN MAGNETIC RESONANCE TOMOGRAPHY

(71) Applicant: Siemens Healthcare GmbH, Erlangen (DE)

(72) Inventors: David Grodzki, Erlangen (DE); Dieter Ritter, Fürth (DE); Patrick Liebig, Erlangen (DE); Rainer Schneider, Höchstadt (DE)

(21) Appl. No.: 17/941,672

(22) Filed: Sep. 9, 2022

(30) Foreign Application Priority Data
Sep. 15, 2021 (EP) 21196793.0

(51) Int. Cl. G01R 33/48; A61B 5/055

(52) U.S. Cl. CPC G01R 33/4828; A61B 5/055

(57) ABSTRACT

In order to improve fat saturation in magnetic resonance technology (MRT) methods, a method for spectral saturation that includes specifying or ascertaining a first resonance frequency of a first substance and a first saturation frequency for a second substance is provided. A saturation pulse that causes no saturation of the first substance at the first resonance frequency is generated. The saturation pulse has a first spectral peak for saturation of the second substance at the first saturation frequency and a second spectral peak at a second saturation frequency. This allows a widening of a spectral saturation bandwidth of a dynamic saturation.

This application claims the benefit of European Patent Application No. EP 21196793.0, filed on Sep. 15, 2021, which is hereby incorporated by reference in its entirety.

### BACKGROUND

The present embodiments relate to generating a saturation pulse for spectral saturation in magnetic resonance tomography, and imaging in magnetic resonance tomography.

Magnetic resonance tomography (MRT) is an imaging method that is used primarily in the field of medical diagnostics. MRT uses a strong and homogenous static magnetic field, also referred to as the B₀ field. In MRT, the relaxation times of nuclear spins are observed as a reaction to a radiofrequency (RF) excitation pulse, in particular the longitudinal relaxation time T₁ and the transverse relaxation time T₂. Most important in this case is the measurement of relaxation times of the hydrogen nucleus (e.g., a proton) due to its frequency of occurrence in the human body.

One requirement in MRT is to generate a good contrast between fatty tissue and aqueous tissue, and, for example, to minimize interference effects of fat signals in aqueous tissue. Separation of the signals of fat molecules and water molecules is possible due to the different chemical shift of the two signals. A chemical shift may be a shift in the resonance frequency of a nucleus as a function of its electrical and chemical environment.

One method for suppressing fat signals based on the different chemical shift of hydrogen nuclei in the fatty tissue and in aqueous tissue is known as spectral fat saturation. In a magnetic resonance (MR) spectrum, the resonances of hydrogen nuclei in fat molecules and water molecules appear as separate signals in the form of a fat peak and water peak. A spectrally selective RF excitation pulse that only excites the hydrogen nuclei in the fatty tissue is transmitted, such that the longitudinal magnetization in the fat is converted into a transverse magnetization. This is immediately dephased by a magnetic field gradient, such that the fatty tissue may no longer be represented by the immediately following imaging sequence.

The spectral fat saturation is, however, susceptible to inhomogeneities of the B₀ field. These specifically determine the position and the width of the fat peak in the MR spectrum. This dependency therefore provides that the quality of the fat signal suppression may be distributed in a spatially inhomogeneous manner. One option for adjusting the homogeneity of the B₀ field is known as “shimming”. Most MRT systems therefore have shim coils that are even able to correct more complex spatial profiles of magnetic fields.

The publication “Broadband Slab Selection with B₁⁺ Mitigation at 7T via Parallel Spectral-Spatial Excitation” (Setsompop et al., MRM 2009) discloses multi-channel applications. A range of excitation frequencies are used for PTX layer excitation or slab excitation (e.g., parallel transmission mode).

The publication DE 10 2012 214 660 B4 discloses an automated spectral fat saturation. The method is based on recording an MR spectrum with the aid of an RF coil and an MR device, a static magnetic field B₀ being present in the recording volume of the MRT device, and automatic analysis of the MR spectrum by searching for two resonance signals in the form of a fat peak and a water peak, and a minimum between these two peaks. Such an automatic analysis is used to automatically decide, as a function of a criterion that may be specified, whether to perform a series of acts that includes adjusting the homogeneity of the static magnetic field B₀ with the aid of a shim coil of the MRT device and calculating an RF pulse for fat saturation as a function of the result of the above search.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the effort required to calculate a saturation pulse may be reduced.

According to the present embodiments, a method is therefore provided for generating a saturation pulse for spectral saturation in magnetic resonance tomography. The spectral saturation is intended to suppress the signal of a specific substance from an MRT signal. The method may therefore be part of a magnetic resonance application (e.g., MR imaging). For example, the method is intended to suppress a substance that interferes with the imaging or otherwise interferes with the MRT signal. In a specific example, the method may be intended to suppress fatty tissue, in which case the method may be used for spectral fat saturation.

In an act (e.g., a first act) of the method, a first resonance frequency of an MR spectrum of a first substance is specified or ascertained. This first substance therefore has a first resonance frequency in the MR spectrum. Such a resonance frequency may be found at the location of a spectral peak, for example. This resonance frequency may be known from the outset and therefore specified. Alternatively, the resonance frequency may also be ascertained manually or automatically. In either case, it is then known where the first substance produces a maximum in the MR spectrum.

Similarly, in a further act, a first saturation frequency is specified or ascertained in a predefined range around a second resonance frequency of the MR spectrum of a second substance. The second substance causes a second spectral peak or a second maximum in the MR spectrum. This maximum is situated at the location of the second resonance frequency. A first saturation frequency with which it is intended to saturate the second substance is now ascertained or specified (e.g., manually or automatically) for the second substance. The predefined range in which the first saturation frequency lies is derived either from a prior measurement or, for example, from experience or similar. For example, typical inhomogeneities of the B₀ field result in known shifts of a fat peak. In this case, the first saturation frequency may lie at the resonance frequency of the shifted fat peak. The range may be very small and even nil in the extreme case. Further, the range may also be defined relative to the first resonance frequency (e.g., −3.0 to −3.5 ppm). The range may be defined by outer limits of the range alone (e.g., −3.0 ppm and −3.5 ppm).

For the saturation, within the context of the method of the present embodiments, a saturation pulse may be generated. This generation of the saturation pulse includes not only the arithmetic determination of the parameters of the pulse, but also the physical transmission of this saturation pulse. This saturation pulse is specified such that the saturation pulse causes no saturation of the first substance at the first resonance frequency. Since the MR spectrum of even a single compound is not a line spectrum but a distribution spectrum, the MR spectrum has a value other than nil at every frequency. These values are, however, negligible at a certain distance from the resonance frequency. For example, fat also has a small spectral component at the resonance frequency of water, but this is usually negligible. In practice, it is assumed that the saturation pulse causes no saturation of the first substance at the first resonance frequency. This provides that the first substance returns practically no MR signal immediately after the saturation pulse.

Further, the saturation pulse is configured so as to have a first spectral peak for saturation of the second substance at the first saturation frequency. While the first substance therefore cannot be saturated by the saturation pulse, the second substance is saturated during excitation by the saturation pulse, at least under certain conditions (e.g., of the B₀ field). The saturation level of the second substance may be 80 to 100%, where 100% may represent total saturation. Such total saturation is always desirable as a rule, but is usually impossible to achieve. The saturation pulse therefore has a maximum at the first saturation frequency and as low a value as possible at the first resonance frequency.

In addition, provision is made for the saturation pulse to have a second spectral peak for saturation of the second substance at a second saturation frequency, which differs from the first saturation frequency, in the predefined range. The saturation pulse therefore has at least two spectral peaks for saturation of the second substance (e.g., for two different conditions in relation to the homogeneity of the B₀ field) and, at the position of the first resonance frequency, a spectral component that causes no saturation of the first substance. It is then possible using two spectral peaks to selectively produce a saturation of the second substance under different conditions. By virtue of the second spectral peak, it is therefore possible to accomplish a saturation of the second substance even if, for example, the MR resonance frequency of the second substance shifts due to a variation of the B₀ field. As a consequence, the second substance may be saturated more reliably. For example, if fat saturation is desired, the second fat saturation peak makes it possible to increase the level of the fat saturation, such that fatty tissue therefore appears darker in an MR image, for example, and does not conceal other tissue sections not based or less based on fatty tissue. Using this second spectral peak, it is therefore possible to deactivate an MR peak of the second substance more selectively.

This has the particular advantage that fluctuations in the B₀ map for a slice of an examined object do not overly affect the imaging as a result of saturation of the second substance (e.g., fat saturation). A second, third, fourth, etc. spectral peak in the saturation pulse for saturation of the second substance actually increases the probability that the second substance will also be saturated to a high degree (e.g., 100%). At the same time, however, this also has the advantage that fluctuations of the B₀ field from slice to slice become increasingly less apparent due to the plurality of spectral peaks for saturation of the second substance. This provides that a saturation pulse may be used not only for a single slice, but also for one or more adjacent slices if applicable.

In an exemplary embodiment, in addition to the first and second spectral peaks, the saturation pulse has, as suggested above, at least one further spectral peak for saturation of the second substance. The saturation pulse therefore has at least three spectral peaks in this case, in order to achieve a saturation of the second substance. In this way, a plurality of spectral components of the second substance may be selectively deactivated in the MR method. The width of the individual spectral peak may also be changed if applicable.

In an embodiment variant, the first substance is water, and the second substance is fat. Using the method according to the present embodiments, it is therefore intended to provide the means by which a high fat saturation may be achieved while structures based on water compounds are to remain unsaturated. It is thereby possible to provide, particularly in the context of MR imaging, that structures of an organism that are based on water compounds are not concealed by fatty tissue. Since the fatty tissue in this case returns little or no MR signal, the fatty tissue appears dark, such that even tissue components that only return a weak signal may be made more visible.

In a further embodiment variant, provision is made for each spectral peak of the saturation pulse to correspond to a peak of an MR spectrum of the second substance. This is particularly beneficial if, for example, the second substance has a plurality of peaks in the MR spectrum. In this case, the second substance may return a plurality of components in the MR signal, which is to be individually saturated. This provides that more extensive saturation of the second substance may ultimately be achieved.

In an embodiment of the method, before the generation of the saturation pulse, a spatially resolved B₀ field map of a magnetic static field of the magnetic resonance tomography is ascertained, and as a function of the spatially resolved B₀ field map, a spectral shift of the saturation pulse is effected at respective locations of the B₀ field map. The B₀ field map may be ascertained as a function of B₀ variations induced by a patient. The B₀ field map may also be ascertained as a function of dynamic effects caused by gradient fields at the time of the excitation pulse. The B₀ field map contains data relating to spatial variations of the B₀ field in the recording region of the magnetic resonance tomography system. Unlike the B₁ field, the B₀ field is considered to be a magnetic field that only has time-relative variations at frequencies significantly lower than the Larmor frequency (e.g., by a factor of 10, 50 or more). The B₀ field map may be stored in a memory of the control unit (e.g., if the B₀ field map was already determined during production by measurement with a field camera or if a field distribution for the magnetic resonance tomography system was already ascertained by calculation). The B₀ field map may, however, also be contemporaneously ascertained by the control unit at the beginning of a sequence by a magnetic resonance measurement or by simulation, for example, taking into consideration the position and other properties of the patient or settings of the magnetic resonance tomography system such as shim currents created by shim coils.

According to a development, a parameter value of the magnetic resonance tomography may be determined or automatically ascertained, and a spectral location of the first spectral peak and/or the second spectral peak is ascertained as a function of the parameter value. For example, if the strength of the B₀ field is known exactly at a specific position of a slice, it is then possible to ascertain the spectral location of a peak more precisely, and the saturation pulse may therefore be adjusted more accurately accordingly. The parameter value of the magnetic resonance tomography may, however, also relate to a specific region of the object to be examined (e.g., human body). A specific body region may therefore be determined by the parameter value, for example. For example, the spectral peak for fat in the head region lies at a different resonance frequency than in the neck region. If the corresponding region of the body or object is now determined by the parameter value, it is then also possible to set the location of the spectral peak or saturation peak more accurately. The parameter value may, however, also relate to other environment variables that influence the MR spectrum and therefore the saturation peak.

In a further development of the method of the present embodiments, a volume that is to be examined by magnetic resonance tomography is clustered into two regions in relation to the first substance and the second substance, and the saturation pulse has the first spectral peak and the second spectral peak for one of the regions and only a single spectral peak for the other region. For example, a body may be divided into regions that predominantly have a fat component or predominantly have a water component. It is thus possible to determine a water region and a fat region. For the water region with only a small fat component, it may be sufficient for the saturation pulse to have only a single spectral peak. Less effort is required to calculate such a saturation pulse. By contrast, in the fat region where the fat component is predominant, it may be beneficial to configure the saturation pulse with two or more spectral peaks for the fat saturation. The calculation of the saturation pulse for the fat region requires rather more effort accordingly. However, since it is not necessary to use a plurality of spectral peaks for the fat saturation in both regions, computing time may be saved in total for the calculation of the saturation pulse over all regions.

The first and second spectral peaks in one of the regions (e.g., fat region) may have a different resonance frequency from the single spectral peak for the other region (e.g., water region). Therefore, for example, the spectral peak for the fat saturation in the water region may be at −3.4 ppm, while the two spectral peaks for fat saturation in the fat region may be at −3.0 ppm and −3.6 ppm. Using the two spectral peaks, it is therefore possible to achieve fat saturation in a wider spectral range. This is important for the fat region, in particular, since it would otherwise be dominated by the fat signals in the MR image.

The present embodiments also relate to a method for imaging in the field of magnetic resonance tomography. This method includes generation of a magnetic static field (e.g., the B₀ field). An excitation pulse is then generated including a saturation pulse that is generated according to the method described above. In the present case, the excitation pulse is therefore considered as the overall pulse, this having a plurality of alternating components. High-frequency components of the excitation pulse are used to obtain corresponding MR signals from the corresponding relaxations of the spin precessions, and high-frequency saturation components of the excitation pulse (e.g., the saturation pulse) are used to scatter the MR signals of a second substance (e.g., fat), so that, for example, these regions of the object to be examined do not conceal the structures that are sought. Finally, for the purpose of imaging, a magnetic resonance signal is captured as a response to the magnetic static field and the excitation pulse (e.g., including saturation pulse), and an image is generated from the magnetic resonance signal. As a result of the saturation of the second substance (e.g., fat), signals from the second substance do not conceal the MR signals from other substances.

In a specific embodiment of the imaging method, provision is made for capturing an image for each slice of a plurality of adjacent slices of an object that is to be examined, and for the same saturation pulse to be used in each case for the plurality of adjacent slices even though different static field distributions are present in the slices. A single saturation pulse is therefore used for a plurality of slices, even if different static field distributions are present in these slices. The static field distributions may differ not only regionally, but the mean values of the static field in the individual slices may also differ. As a result of the plurality of saturation peaks in the saturation pulse, adequate saturation of the second substance may be achieved in all slices despite the differences in static field. It is therefore not necessary to calculate a separate saturation pulse for each slice.

In a development of this, provision is made for automatically deciding that a saturation pulse will be used for the plurality of adjacent slices if a variation of the static field in the plurality of adjacent slices is less than a specified amount or less than an amount that is proportional to the spectral width of the saturation pulse. It may therefore be automatically determined whether the saturation pulse may be used for a plurality of slices and, if applicable, also for how many slices. This decision may be made as a function of the regional change in the static field within a slice and/or the change in the static field from slice to slice. Corresponding threshold values may be used for the decision.

Further, a computer program product that may be invoked from an internal memory of a computer and includes a computer program for performing the acts of a method cited above is provided. Provision is therefore additionally made for a computer program having program means that may be loaded directly into a memory unit of a control unit of the MR installation in order to execute the acts of the method as described above or below when the program means are executed in the control unit. Also provided is a computer-readable medium (e.g., an electronically readable data medium on which the computer program product of the above type is executably stored). Electronically readable control information is therefore stored on the data medium. The control information is configured so as to perform the method described above or below when the data medium is used in a control unit of the MR installation.

As another example, a magnetic resonance system includes a data processing facility for specifying or ascertaining a first resonance frequency of an MR spectrum of a first substance, and for specifying or ascertaining a first saturation frequency in a predefined range around a second resonance frequency of the MR spectrum of a second substance. The magnetic resonance system also includes an RF coil for generating a saturation pulse that essentially causes no saturation of the first substance at the first resonance frequency. The saturation pulse has a first spectral peak for saturation of the second substance at the first saturation frequency. The saturation pulse has a second spectral peak for saturation of the second substance at a second saturation frequency, which differs from the first saturation frequency, in the predefined range.

The advantages and developments indicated in connection with the method of the present embodiments described above may be applied analogously to the magnetic resonance system. The magnetic resonance system has corresponding devices, such as the data processing facility and the RF coil, these being configured so as to execute the method acts cited above.

The features set out above and those described in the following may be used not only in the corresponding combinations explicitly set out, but also in further combinations unless explicitly stated otherwise.

### BRIEF DESCRIPTION OF THE DRAWINGS

FIG. 1 shows a magnetic resonance (MR) spectrum with spectral peaks for the purpose of saturation;

FIG. 2 shows a schematic flow diagram of an exemplary embodiment of a method;

FIG. 3 shows a magnetic resonance tomography (MRT) image of a back with standard fat saturation;

FIG. 4 shows a measured B₀ field map;

FIG. 5 shows an MRT image of a back with fat saturation according to an embodiment;

FIG. 6 shows an MRT image of a cranium with standard fat saturation;

FIG. 7 shows an MRT image of a cranium with fat saturation according to an embodiment; and

FIG. 8 shows the basic structure of an MRT system.

### DETAILED DESCRIPTION

The exemplary embodiments described in detail below represent embodiment variants. Identical or similar elements in the figures are designated by same reference signs. The figures are also schematic illustrations of various embodiment variants. The elements represented in the figures are not necessarily illustrated to scale. Rather, the elements are depicted such that function and purpose of the elements may be understood by a person skilled in the art. The connections illustrated in the figures between functional units or other elements may also be implemented as indirect connections, where a connection may be wireless or wire-based. Functional units may be implemented as hardware, software, or a combination of hardware and software.

The present embodiments have, as a starting point, a method for spectral saturation of matter or substances such as fat or water. Variations of the magnetic static field (e.g., B₀ field) in the image region, caused, for example, by the system (e.g., magnet, eddy currents) or the patient (e.g., anatomy, for example in the neck), are taken into consideration so that the correct target frequency is saturated at each location point. The target frequency is shifted relative to B₀ if necessary. The method is based first on the determination of the B₀ field map (see FIG. 4) using measurement and/or simulation; the method is based second on the dynamic excitation pulses that are calculated, for example, in a PTX framework (e.g., parallel transmission technology).

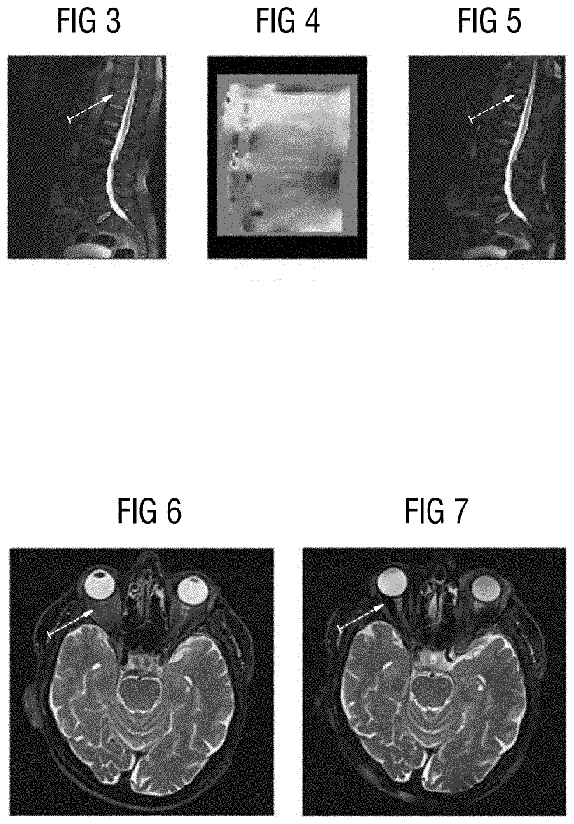

The problems of conventional fat saturation are shown in FIGS. 3 and 4. FIG. 3 shows a magnetic resonance tomography (MRT) image of the back of a patient. Good fat saturation may be achieved in a central region of the spine, as shown by the dark regions that allow lighter structures to be identified. However, in the upper region of the image (see arrow), the fat saturation is inadequate. The fatty tissue returns MR signals, thereby producing gray regions that conceal structures of the spine. This is caused by changes in the B₀ field or inhomogeneities that are system-related or produced by the patient concerned.

FIG. 4 shows a B₀ field map from which the inhomogeneities are evident. For example, clear variations from the average magnetic field strength of the B₀ field are apparent in the upper left-hand region of the B₀ field map. These result in a shift of the fat peak in the MR spectrum, and therefore, only low fat saturation may be achieved in the corresponding image region. Until now, the objective has therefore always been to generate a largely error-free B₀ field map as a basis for calculating the excitation pulse or saturation pulse.

The present embodiments provide an alternative approach, by which both the influence of variations in the B₀ field map may be reduced and further use of a pulse (e.g., abbreviation for excitation pulse including saturation pulse) may be achieved in adjacent slices.

Until now, only two spectral boundary conditions were specified for pulse calculation: For water protons, no saturation (0%) is to occur at the Larmor frequency (0 Hz variation); and for fat-bound protons, total saturation (100%) may occur at −3.4 ppm relative to the resonance frequency of the water protons (e.g., abbreviated to water).

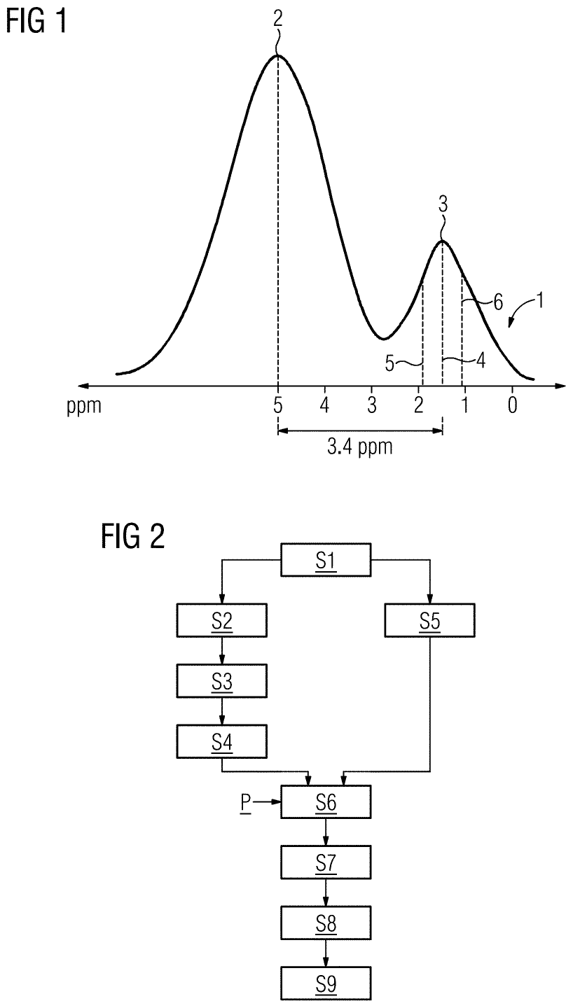

Regarding this, FIG. 1 shows an MR spectrum 1 of water and fat. Water returns a spectral peak 2 at approximately 4.9 ppm on the illustrated scale, while the spectral peak 3 of fat lies at approximately 1.5 ppm. Both peaks 2 and 3 therefore have a separation of approximately 3.4 ppm. These values relate to identical values of the B₀ field in both the water region and the fat region. If, however, the values of the magnetic static field B₀ differ in the water region and in the fat region, the two peaks 2 and 3 may shift relative to each other. Therefore, in the case of a homogeneous magnetic field, a total fat saturation may be achieved with a saturation pulse at −3.4 ppm relative to the resonance frequency of the water protons, but in the case of an inhomogeneous magnetic field, if the resonance frequency of the fat peak 3 no longer has a separation of 3.4 ppm from the resonance frequency of the water peak 2, it is not possible to achieve full saturation with a saturation peak at −3.4 ppm. The aim may, however, be to achieve at least 80 to 100% saturation of the fat (e.g., generally the second substance). For this reason, the spectral width of the saturation pulse is increased.

In FIG. 1, for the case of the homogeneous magnetic field B₀, a fat saturation pulse 4 is shown with a spectral peak at −3.4 ppm relative to water. The saturation peak 4 is marked in simply as a spectral line, but is intended nonetheless to represent a spectral peak. In order to guard against a shift of the fat spectrum, the saturation pulse is widened by adding a further spectral peak 5 (e.g., at −3.0 ppm (as a further saturation frequency)). The further spectral peak 5 or the further saturation frequency is therefore situated, for example, in a predefined range of 0.4 ppm around the resonance frequency of the fat peak 3. If the spectral peak 3 of fat then moves closer to the spectral peak 2 of water due to an inhomogeneity of the B₀ field, it is possible, if applicable by the second saturation peak 5 again, to achieve a higher saturation than may be achieved by the first saturation peak 4. Therefore, for example, the following condition is added for the purpose of calculating the excitation pulse or saturation pulse: For fat-bound protons, total saturation (100%) may occur at −3.0 ppm.

However, in order to cover the case in which the spectral peak 3 of fat drifts further from the spectral peak 2 of the water, the saturation pulse may alternatively or additionally be provided with a third saturation peak 6. This is situated, for example, at −3.6 ppm (e.g., as a second saturation frequency) relative to the spectral peak 2 of the water. Accordingly, the following additional condition may be introduced for the purpose of calculating the excitation pulse or saturation pulse: For fat-bound protons, total saturation (100%) may occur at −3.6 ppm.

In this way, for the purpose of fat saturation (e.g., generally saturation of the second substance), one or a plurality of saturation peaks may be inserted into the saturation pulse at a desired separation from the spectral peak of water (e.g., generally first substance). Specifically, additional saturation peaks at corresponding saturation frequencies may therefore also be introduced between or beyond the two saturation peaks 4, 5.

Fat, which is to be saturated, has peaks in the MR spectrum at −3.3 ppm, −2.5 ppm, +0.7 ppm, −3.7 ppm, −1.8 ppm, and −0.4 ppm relative to water. It is not possible to cover all peaks using conventional fat saturation methods. For this reason, the present method allows the creation of a spectral fat pulse that is able to cover a plurality of peaks or all peaks in this spectrum.

This has the following effects. If erroneous variations are present in the B₀ field map that is used for the pulse calculation, the variations being nonetheless smaller than the “width” of the saturation pulse (e.g., from −3.0 ppm to −3.6 ppm in the example above), greater resilience with respect to fat saturation may be achieved in spite of the variations. For recordings involving a plurality of slices, if different B₀ field maps are present in the slices and their distribution differs between the slices, it is now possible to use a single saturation pulse or excitation pulse for a plurality of slices. This is possible if the variation of the B₀ field at a position does not differ further than the “width” of the saturation pulse. The method also includes an algorithm in this case, that may determine, based on the measured B₀ field maps over a plurality of slices, whether a new pulse calculation is required for a slice. The pulse calculation lasts longer due to the additional boundary conditions. At first approximation, it may be assumed that the computing time increases linearly with the number of spectral boundary conditions. This longer time may nonetheless be balanced against the fact that overall fewer pulses or only one pulse is to be calculated (e.g., in the case of multi-slice measurements).

The method may be embodied such that the spectral width of the overall saturation pulse may either be specified by the user or automatically determined based on further environment variables such as B₀ field strength, body region, etc. For example, a second saturation peak 5 is automatically used in addition to the first saturation peak 4 if the B₀ field has a certain inhomogeneity. If the inhomogeneity is greater, it may be automatically determined that the saturation pulse has a third saturation peak 6. The use of the saturation peaks and their sequence may be selected as desired.

A static B₀ field map of the MR scanner is optionally ascertained at least for the examination volume to be scanned. The B₀ field map may be stored for example in a memory of the control unit for the magnetic resonance tomography system and retrieved from there by the control unit. However, retrieval from an external memory or via a network may also be provided.

The B₀ field map may already be available as a result of for example simulation at the time of configuration or measurement with a field camera during the production process.

Additionally or alternatively, before the measurement, the control unit may measure a B₀ field map using, for example, a rapid sequence in order to show the B₀ changes caused by the patient, at least in the examination volume. The control unit itself may also provide the B₀ field map using simulation, using simplified assumptions if necessary.

As a function of the spatially resolved B₀ field map, it is now possible to effect a spectral shift of the saturation pulse at respective locations of the B₀ field map. This has the effect that the locally variable (e.g., dynamic) saturation pulse compensates for the inhomogeneities of the B₀ field.

In a further embodiment variant, the object to be examined or the B₀ field map may also be spatially clustered into fat regions (e.g., voxels >50% fat) and water regions (e.g., voxels >50% water). The spectral expansions described above may then be restricted to the fat regions. This provides that an exemplary third condition and an exemplary fourth condition may be a combination of a spectral concurrence of total saturation at −3.4 ppm for water regions and total saturation at −3.0 ppm and −3.6 ppm for fat regions. The available spatial-spectral degrees of freedom are thereby concentrated primarily on the problematic zones, instead of being used for spatially global suppression in each case. A fat mapping or water mapping may be derived from an existing Dixon measurement or, for example, from a B₀ mapping sequence.

FIG. 2 schematically shows the execution of a method in an embodiment variant. For this, provision is initially made in an optional act S1 for clustering the examination region or the B₀ field map into a region of the first substance (e.g., water) and a region of the second substance (e.g., fat). For the regions of the second substance (subsequently designated as fat), provision is made in act S2 for specifying or ascertaining a first resonance frequency of the MR spectrum of the water (subsequently designating the first substance).

In a further act S3, provision is made for specifying or ascertaining a first saturation frequency in a predefined range around a second resonance frequency of the MR spectrum of fat (this subsequently designating the second substance). In an optional act S4 following thereupon, a second saturation frequency in the predefined range is specified or ascertained (e.g., by measurement) for the purpose of saturating the fat.

Depending on the clustering in act S1, the method may jump to act S5 instead of jumping to act S2. In act S5, only a single saturation frequency or a single saturation peak is specified or ascertained. For example, in a water region of the object to be examined, it is sufficient to use a single fat peak for the purpose of fat saturation.

In act S6, which follows act S4 or act S5, a saturation pulse that may be part of an excitation pulse is generated. This saturation pulse causes no saturation of the water at the first resonance frequency of the water. In addition, the saturation pulse has a first spectral peak (e.g., first saturation peak at a first saturation frequency) for saturation of the fat at the first saturation frequency (e.g., a resonance frequency of the fat). Finally, the saturation pulse also has a second spectral peak (e.g., second saturation peak at a second saturation frequency) for saturation of the fat at the second saturation frequency in the predefined range. The second saturation frequency is different than the first saturation frequency. If required, one or more parameters P of the MRT system or from the environment (e.g., of the object to be examined) are also taken into consideration for the purpose of generating the saturation pulse in act S6. In a next act S7, provision is made for generating an excitation pulse for the MR analysis. This act S7 may take place jointly with the act S6.

In act S8 following thereupon, provision is made for capturing a magnetic resonance signal as a response to the excitation pulse or saturation pulse. Finally, an image is generated from the magnetic resonance signal in act S9. For the present embodiments, however, only the acts S2, S3, and S6 are of primary significance.

FIGS. 3 to 5 show the advantage of the method of the present embodiments with reference to MRT imaging of the spine. As explained above in connection with FIGS. 3 and 4, certain structural regions in the region of the arrow in FIG. 3 are concealed by fatty tissue, since only a conventional fat saturation method was used there. In the example of FIG. 5, however, use was made of an additional spectral peak for the purpose of fat saturation in the method according to the present embodiments. In this case, the structural regions of the spine at the arrow marked in FIG. 5 may be identified more easily because a high fat saturation was achieved there.

This may be confirmed similarly by the cranial recordings in FIGS. 6 and 7. For the recording in FIG. 6, conventional fat saturation was used. For the recording in FIG. 7, however, the fat saturation of the present embodiments was used with at least one further spectral peak. As shown by the eye sockets indicated by arrows, fat content in the tissue behind the eye conceals the optic nerve. In FIG. 7, however, the optic nerve may easily be identified because the optic nerve is not concealed by fatty tissue in the eye sockets.

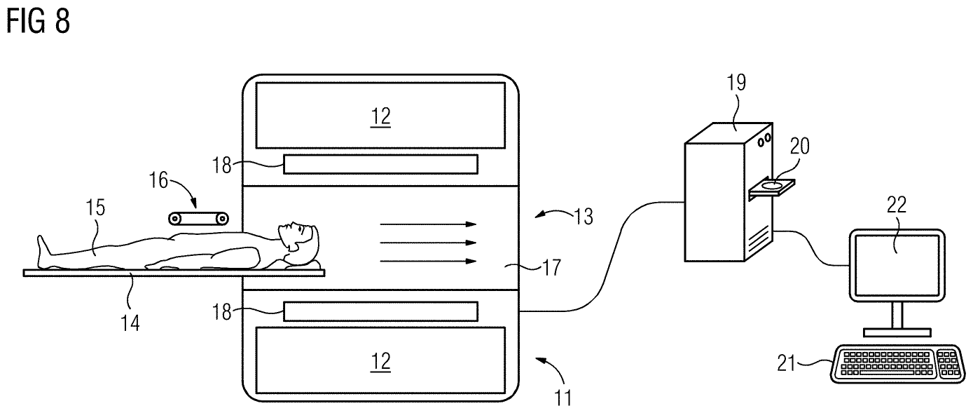

FIG. 8 shows a cross section of an exemplary embodiment of a magnetic resonance system for improved spectral fat saturation, including an MRT device 11, a control unit or data processing facility 19, an input unit 21, and an output unit 22. A computer-readable medium 20 (e.g., DVD, USB stick, or similar) may be processed by the data processing facility 19. For example, stored on the computer-readable medium 20 is a computer program by which the acts of the method illustrated in FIG. 2 may be initiated or controlled. The data processing facility 19 is configured accordingly in order to control or perform these method acts.

The MRT device 11 in the following example includes a cryostat 12 in which a magnet composed of a superconductive material is situated. Such a cryostat 12 is typically filled with liquid helium in order to cool the magnet to below the transition temperature and take the magnet into the superconductive state. A superconductive magnet is to be provided in order to generate a strong static magnetic field B₀ 17 (e.g., a plurality of tesla) in a large recording volume 13. The cryostat 12 and the magnet are typically configured essentially as a hollow cylinder. The static magnetic field B₀ 17 may be generated in a hollow interior of the hollow cylinder. Further to this, the MRT device 11 has RF coils 18 that surround the recording volume 13.

The patient 15 is moved into the recording volume 13 on a patient couch 14 for the purpose of examination by the MRT device 11. In order to record a tomographic image of the patient 15 using the MRT device 11, a local coil 16 may also be used. The RF coils and/or the local coil 16 are typically used for both transmitting and receiving.

These coils 16 and 18 are also used to transmit the saturation pulses with their saturation peaks or spectral peaks.

As shown by the exemplary embodiments above, the widening of the spectral saturation bandwidth of the present embodiments allows dynamic saturation because further boundary conditions may be specified for the pulse calculation, defining the saturation effect at desired frequencies and locations. Using this method, the resilience against inhomogeneities in the B₀ field makes it possible to achieve improved stability of the method and faster calculation times for multi-slice measurements if applicable.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for generating a saturation pulse for spectral saturation in magnetic resonance tomography, the method comprising:
specifying or ascertaining a first resonance frequency of a magnetic resonance (MR) spectrum of a first substance;
specifying or ascertaining a first saturation frequency in a predefined range around a second resonance frequency of the MR spectrum of a second substance; and
generating a saturation pulse that causes no saturation of the first substance at the first resonance frequency,
wherein the saturation pulse has a first spectral peak for saturation of the second substance at the first saturation frequency, and
wherein the saturation pulse has a second spectral peak for saturation of the second substance at a second saturation frequency in the predefined range, the second saturation frequency differing from the first saturation frequency.

2. The method of claim 1, wherein the saturation pulse has, in addition to the first spectral peak and the second spectral peak, at least one further spectral peak for saturation of the second substance.

3. The method of claim 1, wherein the first substance is water, and the second substance is fat.

4. The method of claim 1, wherein each spectral peak of the saturation pulse corresponds to a peak of an MR spectrum of the second substance.

5. The method of claim 1, wherein before generating the saturation pulse, the method further comprises:
ascertaining a spatially resolved B₀ field map of a magnetic static field of the magnetic resonance tomography; and
effecting a spectral shift of the saturation pulse at respective locations of the B₀ field map as a function of the spatially resolved B₀ field map.

6. The method of claim 1, further comprising:
determining or automatically ascertaining a parameter value of the magnetic resonance tomography; and
automatically ascertaining a spectral location of the second spectral peak as a function of the parameter value.

7. The method of claim 6, wherein the parameter value relates to a fluctuation in a field strength of a magnetic static field or a region of an object that is to be examined by the magnetic resonance tomography.

8. The method of claim 1, wherein a volume that is to be examined by the magnetic resonance tomography is clustered into two regions in relation to the first substance and the second substance, and
wherein the saturation pulse has the first spectral peak and the second spectral peak for one of the two regions and only a single spectral peak for the other of the two regions.

9. The method of claim 8, wherein the single spectral peak differs from the first spectral peak and the second spectral peak with respect to a resonance frequency.

10. A method for imaging in magnetic resonance tomography, the method comprising:
generating a magnetic static field;
generating an excitation pulse having a saturation pulse for spectral saturation in the magnetic resonance tomography, generating the saturation pulse comprising:
specifying or ascertaining a first resonance frequency of a magnetic resonance (MR) spectrum of a first substance;
specifying or ascertaining a first saturation frequency in a predefined range around a second resonance frequency of the MR spectrum of a second substance; and
generating the saturation pulse that causes no saturation of the first substance at the first resonance frequency, wherein the saturation pulse has a first spectral peak for saturation of the second substance at the first saturation frequency, and
wherein the saturation pulse has a second spectral peak for saturation of the second substance at a second saturation frequency in the predefined range, the second saturation frequency differing from the first saturation frequency;
capturing a magnetic resonance signal as a response to the magnetic static field and the excitation pulse; and
generating an image from the magnetic resonance signal.

11. The method of claim 10, further comprising capturing an image for each slice of a plurality of adjacent slices of an object that is to be examined,
wherein a same saturation pulse is used in each case for the plurality of adjacent slices, even though different static field distributions are present in the plurality of adjacent slices.

12. The method of claim 10, further comprising automatically deciding that a saturation pulse will be used for the plurality of adjacent slices when a variation of the static field in the plurality of adjacent slices is less than a specified amount or less than an amount that is proportional to a spectral width of the saturation pulse.

13. In a non-transitory computer-readable storage medium that stores instructions executable by one or more processors to generate a saturation pulse for spectral saturation in magnetic resonance tomography, the instructions comprising:
specifying or ascertaining a first resonance frequency of a magnetic resonance (MR) spectrum of a first substance;
specifying or ascertaining a first saturation frequency in a predefined range around a second resonance frequency of the MR spectrum of a second substance; and
generating a saturation pulse that causes no saturation of the first substance at the first resonance frequency,
wherein the saturation pulse has a first spectral peak for saturation of the second substance at the first saturation frequency, and
wherein the saturation pulse has a second spectral peak for saturation of the second substance at a second saturation frequency in the predefined range, the second saturation frequency differing from the first saturation frequency.

14. The non-transitory computer-readable storage medium of claim 13, wherein the saturation pulse has, in addition to the first spectral peak and the second spectral peak, at least one further spectral peak for saturation of the second substance.

15. The non-transitory computer-readable storage medium of claim 13, wherein the first substance is water, and the second substance is fat.

16. The non-transitory computer-readable storage medium of claim 13, wherein each spectral peak of the saturation pulse corresponds to a peak of an MR spectrum of the second substance.

17. The non-transitory computer-readable storage medium of claim 13, wherein before generating the saturation pulse, the instructions further comprise:
ascertaining a spatially resolved B₀ field map of a magnetic static field of the magnetic resonance tomography; and
effecting a spectral shift of the saturation pulse at respective locations of the B₀ field map as a function of the spatially resolved B₀ field map.

18. The non-transitory computer-readable storage medium of claim 13, wherein the instructions further comprise:
determining or automatically ascertaining a parameter value of the magnetic resonance tomography; and
automatically ascertaining a spectral location of the second spectral peak as a function of the parameter value.

19. A magnetic resonance system comprising:
a data processing facility configured to:
specify or ascertain a first resonance frequency of a magnetic resonance (MR) spectrum of a first substance; and
specify or ascertain a first saturation frequency in a predefined range around a second resonance frequency of the MR spectrum of a second substance; and
a radio frequency (RF) coil configured to generate a saturation pulse that causes no saturation of the first substance at the first resonance frequency,
wherein the saturation pulse has a first spectral peak for saturation of the second substance at the first saturation frequency, and
wherein the saturation pulse has a second spectral peak for saturation of the second substance at a second saturation frequency in the predefined range, the second saturation frequency differing from the first saturation frequency.
